(19) United States
(12) Patent Application Publication
(10) Pub. No.: US 2023/0307129 A1
(43) Pub. Date: Sep. 28, 2023

(54) LEARNING DATA GENERATION DEVICE, METHOD FOR DRIVING SAME DEVICE, AND COMPUTER-READABLE RECORDING MEDIUM

(71) Applicant: MEGAGEN IMPLANT CO., LTD., Daegu (KR)

(72) Inventors: Tae Gyoun LEE, Daegu (KR); Boo Kyeong CHOI, Daegu (KR); Jae Hong SO, Changwon-si, Gyeongsangnam-do (KR)

(21) Appl. No.: 18/014,141

(22) PCT Filed: Jun. 3, 2021
(86) PCT No.: PCT/KR2021/006963

§ 371 Date: Dec. 30, 2022

(30) Foreign Application Priority Data
Jul. 8, 2020 (KR) 10-2020-0084069

(51) Int. Cl. G16H 50/20

(52) U.S. Cl. CPC G16H 50/20

(57) ABSTRACT

Disclosed are a training data generation device, a method for driving the same, and a computer-readable recording medium. The training data generation device according to an embodiment of the present inventive concept includes a data receiver configured to receive, as a feedback, a training result output by applying, to a learning model, medical data generated by pre-processing an assigned amount of pathological images; and a controller configured to secure training data for artificial intelligence (AI) or image analysis by correcting medical data of pathological images having errors checked based on the training result received as a feedback, and then reapplying the corrected medical data to the learning model.

### TECHNICAL FIELD

The present inventive concept relates to a training data generation device, a method of driving the same, and a computer-readable recording medium, and more particularly, to a training data generation device, a method for driving the same, and a computer-readable recording medium, in which, for example, during the application of artificial intelligence or an image analysis model by a supervised learning method, errors are corrected by applying a small scale of training data to a learning model to validate data, thereby generating error-free robust training data.

### BACKGROUND ART

Supervised learning means a machine learning method to establish a target model for performing a target task by training a data set with label information (that is, correct answer information). Accordingly, in order to perform supervised learning on a data set without label information (e.g., indicated by a tag icon), annotation, that is, an annotation task, essentially precedes.

The annotation task means a task of tagging label information for each data to generate a training data set. Because the annotation task is generally performed by a human, a considerable amount of human cost and time is consumed to generate a large amount of a training data set. In particular, when a machine learning model to diagnose the type, location, or the like of a lesion in a pathological image is established, the annotation task should be performed by a skilled medical specialist, and thus much more expenses are consumed compared with other domains.

Conventionally, the annotation task has been performed without a systematic task process. For example, according to a conventional method, a manager checks the characteristics of each pathological image with the naked eye, determines whether to perform annotation, manually classifies the pathological image, and then assign the pathological image to appropriate task performer (annotator).

In addition, conventionally, a manager specifies an annotation area on a pathological image, one by one, and then assigns a task to a task performer, In other words, conventionally, the overall process of classifying pathological images, assigning tasks, specifying annotation areas, and the like has been manually performed by a manager, and thus, a considerable amount of time and human cost have been consumed for the annotation task.

Furthermore, in spite that machine learning techniques have been sufficiently advanced, due to the time and cost matters of the annotation task, there have been many difficulties applying machine learning techniques to various fields.

### DETAILED DESCRIPTION OF THE INVENTIVE CONCEPT

### Technical Problem

Provided are a training data generation device, a method for driving the same, and a computer-readable recording medium, in which, for example, during the application of an artificial intelligence or an image analysis model by a supervised learning method, errors are corrected by applying a small scale of training data to a learning model to validate data, thereby generating robust and error-free training data.

### Solution to Problem

According to one aspect of the present inventive concept, a training data generation device includes a data receiver configured to receive, as a feedback, a training result output by applying, to a learning model, medical data generated by pre-processing an assigned amount of pathological images; and a controller configured to secure training data for artificial intelligence (AI) or image analysis by correcting medical data of pathological images having errors checked based on the training result received as a feedback, and then reapplying the corrected medical data to the learning model.

The controller may be further configured to generate a data set for a position of a lesion marked on the pathological images for the pre-processing, and after correcting the generated data set, reapply the corrected data set to the learning model.

The controller may be further configured to perform automatic marking on the pathological images based on previously stored marking information, and correct a position by the automatic marking and apply the corrected position to the learning model.

The controller may be further configured to display, on a screen, a training result received as a feedback to make correction of the data set.

The controller may be further configured to secure new training data when the learning model is changed, by applying the secured training data to the changed learning model.

According to another aspect of the present inventive concept, a method of driving a training data generation device includes receiving, by a data receiver, as a feedback, a training result output by applying, to a learning model, medical data generated by pre-processing an assigned amount of pathological images, and securing, by a controller, training data for artificial intelligence (AI) or image analysis by correcting medical data of pathological images having errors checked based on the training result received as a feedback, and then reapplying the corrected medical data to the learning model.

The securing of the training data may include generating a data set for a position of a lesion marked on the pathological images for the pre-processing, and after correcting the generated data set, reapplying the corrected data set to the learning model.

The method may further include performing, by the controller, automatic marking on the pathological images based on previously stored marking information, and correcting a position by the automatic marking and applying the corrected position to the learning model.

The method may further include displaying, by the controller, on a screen, a training result received as a feedback to make correction of the data set.

The method may further include securing, by the controller, new training data, when the learning model is changed, by applying the secured training data to the changed learning model.

According to another aspect of the present inventive concept, there is a non-transitory computer-readable recording medium having recorded thereon a program for executing a method of driving a training data generation device, the method of driving a training data generation device including receiving, as a feedback, a training result output by applying, to a learning model, medical data generated by pre-processing an assigned amount of pathological images, and securing training data for artificial intelligence (AI) or image analysis by correcting medical data of pathological images having errors checked based on the training result received as a feedback, and then reapplying the corrected medical data to the learning model.

### Advantageous Effects

According to an embodiment of the present inventive concept, during the development of artificial intelligence or image analysis models, the performance of a learning model may be improved by removing errors in training data.

Furthermore, according to an embodiment of the present inventive concept, as training data is generated by applying a small amount of task data (e.g., a pathological image and the like) to a learning model and correcting data while checking a training result, identifying the cause of an error and handling a task may be smoothly performed.

Furthermore, according to an embodiment of the present inventive concept, data optimized according to a selected learning model may be processed.

### DESCRIPTION OF THE DRAWINGS

FIG. 1 illustrates a training data generation system according to an embodiment of the present inventive concept.

FIG. 2 is a block diagram of a detailed structure of a training data generation device of FIG. 1.

FIG. 3 is a block diagram of another detailed structure of the training data generation device of FIG. 1.

FIG. 4 is an example screen implemented by a tool according to an embodiment of the present inventive concept.

FIG. 5 is an example of a pre-processing process performed on the screen of FIG. 4.

FIG. 6 is a flowchart of a driving process of the training data generation device of FIG. 1.

FIG. 7 is a flowchart of another driving process of the training data generation device of FIG. 1.

### MODE OF THE INVENTIVE CONCEPT

In order to fully understand the operational advantages of the present inventive concept and the objectives achieved by the implementation of the present inventive concept, the accompanying drawings illustrating preferred embodiments of the present inventive concept and the contents described in the accompanying drawings are referred to.

Hereinafter, the inventive concept will be described in detail by explaining preferred embodiments of the inventive concept with reference to the attached drawings. Like reference numerals in the drawings denote like elements.

Hereinafter, an embodiment of the present inventive concept is described in detail with reference to the accompanying drawings.

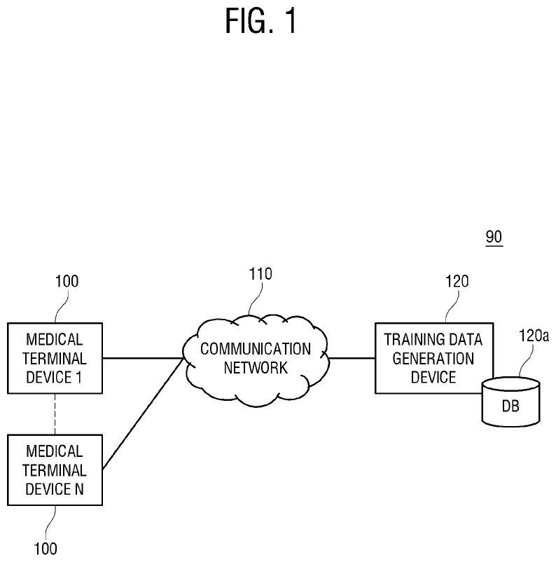

FIG. 1 illustrates a training data generation system according to an embodiment of the present inventive concept.

As illustrated in FIG. 1, a training data generation system 90 according to an embodiment of the present inventive concept may include some or all of a medical terminal device 100, a communication network 110, and a training data generation device 120.

Here, the expression “including some or all” may mean that some constituent elements such as the communication network 110 are omitted so that the medical terminal device 100 and the training data generation device 120 performs a direct (e.g., P2P) communication, some or all constituent elements such as the training data generation device 120 are incorporated into a network device (e.g., a wireless exchange device and the like) constituting the communication network 110, or the like. To help sufficient understanding of the present inventive concept, the following description is based on that all constituent elements are included.

The medical terminal device 100 may include, for example, various devices for providing medical data. The medical terminal device 100 may include, for example, a doctor's computer, a hospital's server, and the like, which is provided in a hospital or related institutes thereof and provides various types of medical data. Moreover, the medical terminal device 100 may have medical data provided by an organization managing public medical data. In addition, the medical terminal device 100 may include various equipment such as MRI equipment for photographing to provide various pieces of medical data. The medical data may include a CT image and the like, which is obtained for cancer diagnosis and the like.

For example, in FIG. 1, when the training data generation device 120 is a server to download a program and the like for training data generation, the medical terminal device 100 according to an embodiment of the present inventive concept may download from the server a program for example, a tool, according to an embodiment of the present inventive concept, and perform a training data generation operation according to an embodiment of the present inventive concept.

Actually, while a training data generation process according to an embodiment of the present inventive concept may be performed by the medical staff of a medical institution, training data optimized for a learning model may be generated by providing medical data to an external professional institution, for example, a program exclusive company and the like, to apply the learning model for artificial intelligence (e.g., CNN, RNN, and the like) or image analysis of a specific function. In an embodiment of the disclosure, the latter case is assumed and described further. As it is also possible that, as in the former case, the operation according to an embodiment of the present inventive concept may be sufficiently performed by medical staff in the medical terminal device 100, the present inventive concept is not limited to any one format.

It is also possible that, to generate robust training data according to an embodiment of the present inventive concept, the medical terminal device 100 may perform a pre-processing operation on medical data such as a CT image and provide data to the training data generation device 120. In an embodiment of the disclosure, it is intended to generate training data in the form of supervised learning, not non-supervised learning, a data set is obtained through an operation in which, for example, a medical expert or an image analysis specialist marks the position of a lesion and the like on a CT image as a lesion image. In doing so, it is requested that the data set generated through marking may enable detection an area through points, lines, surfaces, and the like (by image processing). The data set about a marking area may be generated in the form of coordinate values through pixel analysis.

For example, while according to the related art, to obtain artificial intelligence training data, medical data is provided by performing the pre-processing operation manually by task performers with respect to ten thousands of CT images, in an embodiment of the disclosure, the pre-processing operation such as marking and the like is performed only on, for example, 1000 CT images, and 9000 CT images are provided without marking, that is, the pre-processing operation, so that the time and cost conventionally consumed to generate training data may be considerably reduced. This is because an automatic marking operation or an automatic pre-processing operation according to an embodiment of the present inventive concept is performed. A detailed description thereof is presented below.

The communication network 110 includes both of wired/wireless communication networks. For example, wired/wireless Internet networks may be used or linked as the communication network 110. A wired network includes the Internet network such as a cable network or a public switched telephone network (PSTN), and a wireless communication network includes code division multiple access (CDMA), wideband CDMA (WCDMA), global system for mobile communication (GSM), evolved packet core (EPC), long term evolution (LTE), a Wibro network, and the like. The communication network 110 according to an embodiment of the present inventive concept is not limited thereto, and, for example, a cloud computing network under a cloud computing environment, a 5G network, and the like, may be used as a connection network of a next generation mobile communication system. For example, when the communication network 110 is a wired communication network, an access point in the communication network 110 may be connected to an exchange center of a telephone office and the like, and when the communication network 110 is a wireless communication network, an access point in the communication network 110 may be connected to a serving GPRS support node (SGSN) or a gateway GPRS support node (GGSN) operated by a communication company to process data, or to various repeater such as base station transmission (BTS), NodeB, e-NodeB, and the like to process data.

The communication network 110 may include an access point. The access point includes a small base station such as a femto or pico base station installed in many buildings. The femto or pico base station is classified depending on the maximum number of the medical terminal device 100 and the like, according to the classification of small base stations. The access point may include a short-range communication module to perform a short-range communication, such as Zigbee, Wi-Fi, and the like, with the medical terminal device 100. The access point may use transmission control protocol/Internet protocol (TCP/IP) or real-time streaming protocol (RTSP) for a wireless communication. The short-range communication may be performed in various specifications such as Bluetooth, Zigbee, infrared, radio frequency (RF) such as ultra-high frequency (UHF) and very high frequency (VHF), an ultrawide band communication (UWB), and the like, in addition to Wi-Fi. Accordingly, the access point may extract the location of a data packet, set an optimal communication path for the extracted location, and transmit the data packet to a next device, for example, the training data generation device 120 along the set communication path. The access point may share many lines under a general network environment, and include, for example, a router, a repeater, and the like.

When the medical terminal device 100 is, for example, medical equipment providing a CT image and the like, the training data generation device 120 may include medical staff's computer and the like for collecting medical data from the medical equipment, other medical staff or medical institution's computer and the like for receiving medical data such as a CT image and the like from the medical staff's medical terminal device 100 and the like or medical data that is the CT image on which a pre-processing operation such as marking and the like is performed, and a server or computer of a company who exclusively performs a task according to an embodiment of the present inventive concept. In other words, the training data generation device 120 according to an embodiment of the present inventive concept, as an operating server of a program developer, may supply a program when nationwide medical institutions request the program. Alternatively, the training data generation device 120 may include a computer of a company who performs an operation of collecting medical data from a specific medical institution through the server and then processing the collected data. An embodiment of the present inventive concept is not particularly limited to any one format.

When the medical data such as a CT image and the like is not pre-processed, the training data generation device 120 according to an embodiment of the present inventive concept may perform a pre-processing operation for marking a portion of a lesion and the like on the medical data. Furthermore, when pre-processed medical data is collected, training data to be applied to a specific learning model may be generated by using the medical data. For example, in an embodiment of the disclosure, even when the same data tool for generating training data is used and also the same medical data is used, a training result of the training data, that is, the generated training data, may differ depending on the applied learning model. Accordingly, the training data generation device 120 according to an embodiment of the present inventive concept performs an operation to generate training data optimized for a specific (or set) learning model.

The training data generation device 120 according to an embodiment of the present inventive concept may execute annotation software (SW) capable of establishment of a database for a marking area, division, and detection, between 2D and 3D images and development of artificial intelligence and an image analysis model. Accordingly, an annotation task on the 2D and 3D images is possible, and provided are functions for pre-processing such as the reconstruction of 2D/3D input images, an image filter, a multi planar reconstruction (MPL) operation, and the like. Also, real-time marking, and generation and correction of a region of interest are possible. By using the existing annotation data loading and automatic detection function, an annotation task is possible after previous marker detection, the stored data may be instantly configured as artificial intelligence and image analysis training data, and real-time artificial intelligence and image analysis model learning are possible. Storing and sharing real-time data using a network and a database may be possible.

The type of data used in an embodiment of the present inventive concept includes all medical image (e.g., CT, MRI, X-Ray, ultrasound, nuclear medicine, microscopy, and the like) formats (e.g., DICOM, and the like), all image formats (e.g., JPEG, TIFF, PNG, BMP, SVG, HEIF, GIF, and the like), all three-dimensional data (e.g., STL, OBJ, PLY, and the like). Moreover, the applied field may include extraction of a region of interest in 2D and 3D images (e.g., division of a specific region (e.g., a lesion, a person, an animal, an object, a license plate, a sign plate, and the like) in an image, detection of a landmark in 2D and 3D images (e.g., Cephalo analysis, teeth position, eyes/nose/mouth/chin, and the like), and a function of using a region of interest or landmark (e.g., postoperative prediction, image registration, image correction, analysis report writing, facial expression analysis, virtual simulation, synthesis, and the like).

The training data generation device 120 may perform an operation to effectively perform a data annotation task, and include a program (or tool) therefor and execute the same. Data to train are sequentially called and an area to train is determined and indexed by an expert (e.g., a human). In other words, a person such as a doctor adds annotation. For example, a learning model to be used as an algorithm is transplanted to a tool according to an embodiment of the present inventive concept to check a result, and when there is a portion where a correct answer is not found, the portion is edited and added as training data. In other words, incorrect annotation is partially corrected by a human. By repeating the task, annotation optimized for the transplanted learning model, that is, annotation data may be effectively collected and trained. Although it is a task done by a human, the training data generation device 120 recognizes the task.

In more detail, the training data generation device 120 may add annotation by a supervised learning method by dotting a lesion area or marking an area on thereon on 1000 CT images of ten thousand sheets of medical data, for example, CT images, by a doctor and the like. In an embodiment of the disclosure, this process may be referred to as a data processing operation, and in more detail, a pre-processing operation because data processing is performed prior to the application of a learning model. By executing the tool according to an embodiment of the present inventive concept, that is, a program, 1000 sheets of CT images are applied to a set learning model, or 100 sheets each are applied to the learning model, and thus, a training result may be received as a feedback through a monitor screen and the like so that a checking operation may be performed. This corresponds to an operation prior to the final generation of training data. While an error is checked with a small scale of data unit, data is validated, and also performance is checked, thereby completing collection of training data, that is, a generation operation. As small scaled data is used, error correction may be instantly made.

As described later, after checking the training result through the feedback applied with the learning model, for a CT image in which an error is generated, the data set, that is, data about an error point, is calibrated or corrected, and then by reapplying the learning model, when a true training result is obtained, the training result is obtained as training data and stored in a DB 120a. The previous training result including an error may be erased. While the method according to the related art is a method of reducing the error of training data through an added value, that is, a probability method, to increase accuracy of a typical learning model, that is, a training engine, in other words, technology have been developed by a method of improving a learning model, in the supervised learning method according to an embodiment of the disclosure, by instantly checking a result related to whether training data is insufficient or a supervised learner misplaces a dot, the error of training data is removed so that the time and cost of the task may be reduced. For example, when a user corrects an error on a screen by using a mouse cursor and the like to correct the error, the training data generation device 120 recognizes the correction, and correct data only may be generated or obtained through the above process. For example, the training data used by the learning model according to the related art essentially includes at least 10% false data, and the learning model filters the false data through the probability method, whereas, in an embodiment of the disclosure, the 10% false data included in the training data used by the learning model may be changed closely to 0% through the optimization program described above.

Moreover, the training data generation device 120 according to an embodiment of the present inventive concept does not perform marking, that is, the pre-processing operation, on all of ten thousand sheets of CT images. For example, although a marking operation is performed on one hundred sheets of CT images, after a data set thereof is previously stored, that is, marking information is stored, when a new CT image is received, automatic marking is performed on the received new CT image according to position information of the previously stored data set. As such, when corrections are necessary in a state in which basic setting is made, only some errors are corrected, and thus, a task for the pre-processing operation may be smoothly performed. Then, the training data generation operation may be performed by a method in which a training result, after applied to a learning model, is received as a feedback to check and correct errors again.

In this process, the training data generation device 120 may set the number of, for example, CT images on which the pre-processing operation is performed. In other words, after initially performing marking on only one hundred sheets of images, automatic marking may be performed on CT images received thereafter. Also, it is possible to perform the automatic marking operation after marking is performed on a less number of images, and thus, an embodiment of the disclosure may not be limited particularly to any one method. There may be some difference depending on the performance of a program.

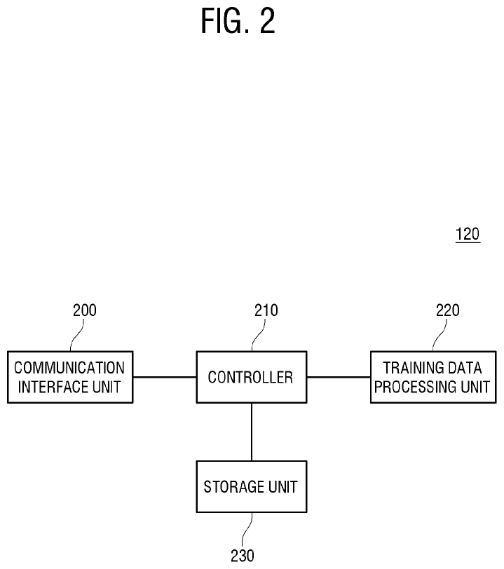

According to the above result, an embodiment of the present inventive concept may considerably reduce the time and cost consumed for generation of training data by a supervised learning method, and also, may considerably reduce or remove error of data used as training data. In other words, as the performance of a learning model is related to high cost, without increasing the performance of a learning model, while reducing the cost for purchasing a learning model, performance may be enhanced. FIG. 2 is a block diagram of a detailed structure of the training data generation device of FIG. 1.

As illustrated in FIG. 2, the training data generation device 120 according to an embodiment of the present inventive concept may include some or all of a communication interface unit 200, a controller 210, a training data processing unit 220, and a storage unit 230, and may further include a display unit.

Here, the expression “including some or all” may mean that some constituent elements such as the storage unit 230 are omitted, some constituent elements such as the training data processing unit 220 are incorporated into another constituent element such as the controller 210, or the like. To help sufficient understanding of the present inventive concept, the following description is based on that all constituent elements are included.

The communication interface unit (or data receiver) 200 may collect medical data by communicating with, for example, the medical terminal device 100 of FIG. 1, or receive medical data on which the pre-processing operation is performed by performing marking on a portion such as a lesion, tumor and the like on the medical data and transmit the medical data to the controller 210. Here, information (e.g., a coordinate value) about the portion in which marking is performed may be referred to as a data set. For example, when a total of 6 points are marked in the portion, coordinate values for the 6 points may become a data set. For example, although the marking on a CT image may be performed through a marker on an image, it may be possible to use a method of previously storing and using only a coordinate value for the point by using a separate masking image. Thus, an embodiment of the disclosure is not limited to any specific method for forming a data set.

The communication interface unit 200 may perform various operations such as modulation/demodulation, muxing/demuxing, encoding/decoding, scaling for converting a resolution, and the like, in a process of transmitting/receiving captured images such as a CT image and the like. The contents related to the above operations are obvious to a person skilled in the art, descriptions thereof are omitted.

The controller 210 handles the overall control operations of the communication interface unit 200, the training data processing unit 220, and the storage unit 230 of FIG. 2. The controller 210 may temporarily store, for example, the medical data received through the communication interface unit 200, in the storage unit 230 and call and provide the medical data to the training data processing unit 220.

Furthermore, the controller 210 may perform various operations for generating training data according to an embodiment of the present inventive concept, in association with the training data processing unit 220. The controller 210, upon a user's request, may control the training data processing unit 220 to execute a built-in data processing optimization program or tool, thereby performing an operation according to an embodiment of the present inventive concept. For example, after performing marking on a CT image or applying a small scale of medical data on which marking is performed to a learning model, a training result thereof is received and displayed on the screen. For a CT image that is determined to have an error, after correcting the error, the CT image is reapplied to the learning model, and after checking a training result and finding that there is no error any more, the CT image is finally secured as training data. The CT images may be systematically classified and stored in the DB 120a of FIG. 1, upon the request of the training data processing unit 220.

Furthermore, when a learning model is changed, the controller 210 may update a program of the training data processing unit 220 to the changed learning model and validate the medical data to check performance thereof, and secure training data. Accordingly, even without using a high performance learning model requiring high cost, the accuracy of learning may be highly improved.

The training data processing unit 220 may include a data receiver, and perform an operation to fundamentally reduce errors in training data generated by applying artificial intelligence or an image analysis model as a supervised learning method according to an embodiment of the present inventive concept. In the supervised learning method, the generation of errors in the training data may be classified into insufficient training data, incorrect data assignment for a portion to find, that is, incorrect annotation, malfunction of a learning model engine, and the like. A considerable time and cost is consumed to finding out the cause of an error by performing marking on, for example, all of ten thousand sheets of CT images and checking a training result thereof, as in a conventional manner. It would be very difficult to find out where the cause is. Accordingly, in an embodiment of the disclosure, after applying a small scale of medical data to a learning model, a result thereof is instantly received as a feedback to check the result, and when there is an error, the error is corrected and then the corrected data is reapplied to the learning model. When the error is corrected, the medical data is secured as training data. Accordingly, the generation of an error in the training data of a supervised learning method is fundamentally prevented.

Furthermore, the training data processing unit 220, when performing marking, that is, a pre-processing operation, in a region of interest such as a lesion in a CT image and the like, may perform automation on the pre-processing operation. For example, when, among ten thousand sheets of CT images, lesions of one hundred sheets of CT image are marked, for example, manually, by an expert such as a doctor and the like, marking is automatically performed from the 101^{st} CT image to the 10000^{th} CT image, and the expert checks the automatically marked positions, that is, data, and then corrects only incorrect portions by using a mouse cursor and the like. The corrected image is applied to the learning model, and when there is an error, the image is corrected again and applied to the learning model. When the error is corrected, medical data of the image is secured as training data. Through the operation, inconvenience and time of a task may be considerably reduced.

Furthermore, when there is a change to a new learning model, an operation to generate new training data may be performed by using the same program according to an embodiment of the present inventive concept. In other words, in an embodiment of the disclosure, although the generation of training data is optimized for a specific learning model, it may not be that such training data is optimized for a different learning model, and in this case, after the learning model is changed, the operation described above is performed again to secure new training data.

The storage unit 230 may store various types of information or data processed under the control of the controller 210 and output the information or data under the control of the controller 210. Typically, the storage unit 230 may temporarily store medical data such as a CT image and provide the medical data to the training data processing unit 220 under the control of the controller 210. Here, as information and data are used interchangeably in practice, the disclosure is not particularly limited to the concept of the term.

Meanwhile, as another embodiment of the present inventive concept, the controller 210 may include a CPU and a memory, and may be formed as a single chip. The CPU may include a control circuit, an arithmetic unit (ALU), an instruction interpretation unit, a registry, and the like, and the memory may include RAM. The control circuit may perform a control operation, the arithmetic unit may perform an arithmetic operation on binary bit information, the instruction interpretation unit may perform an operation of converting high-level languages including an interpreter, a compiler, and the like into a machine language, or a machine language into a high-level language, and the registry may be involved in data storage in software. According to the above configuration, for example, at the initial stage of the operation of the training data generation device 120 of FIG. 1, a program stored in the training data processing unit 220 is copied and loaded in the memory, that is, RAM, and then by executing the program, a data calculation processing speed may be rapidly increased.

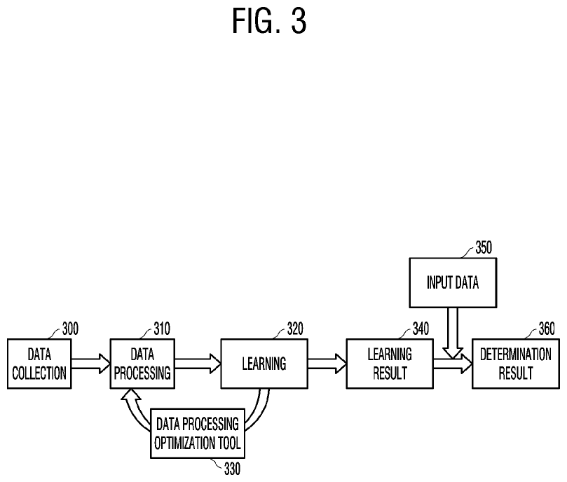

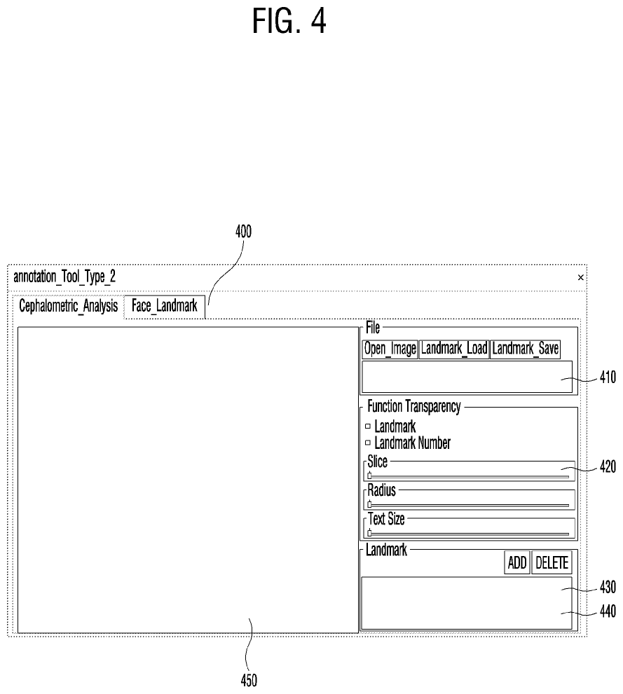

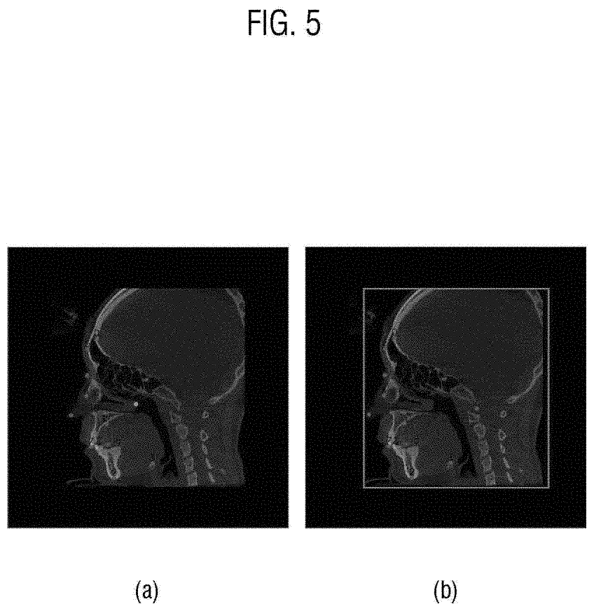

FIG. 3 is a block diagram of another detailed structure of the training data generation device of FIG. 1. FIG. 4 is an example screen implemented by a tool according to an embodiment of the present inventive concept. FIG. 5 is an example of a pre-processing process performed on the screen of FIG. 4.

As illustrated in FIG. 3, a training data generation device 120′ according to another embodiment of the present inventive concept may include some or all of a data collection unit 300, a data processing unit 310, a learning model unit 320, a data processing optimization unit (or an optimization tool) 330, a learning result output unit 340, a data input unit 350, and a determination result output unit 360.

Here, the expression “including some or all” may mean that some constituent elements such as the data input unit 350 are omitted, some constituent elements such as the data processing optimization unit 330 are incorporated into another constituent element such as the learning model unit 320, or the like. To help sufficient understanding of the present inventive concept, the following description is based on that all constituent elements are included. The constituent elements of FIG. 3 according to an embodiment of the present inventive concept may be configured by a hardware (H/W) module, a software (S/W) module, or a combination thereof, and a connection relationship between the respective constituent elements may be changed without limitation.

The data collection unit 300 collect, for example, medical data of CT images. In this process, data marked on the CT images may be collected.

The data processing unit 310 performs a pre-processing operation such as marking and the like on the collected medical data. When the data is marked and collected, the pre-processing operation may be omitted or the existing marking data may be erased and then a new marking operation may be performed. For example, when the data processing unit 310 performs an operation to find a lesion in a CT image, marking may be performed on the lesion.

The data processing unit 310 according to an embodiment of the present inventive concept may perform an operation such as marking and the like in association with the data processing optimization unit 330. In other words, as illustrated in FIG. 4, when a UI screen for marking of the medical data is displayed on the screen by executing a program of the data processing optimization unit 330, the operation such as marking and the like may be performed therethrough. In other words, it may be seen that an operation for generating a data set related to the marking and the like is performed.

The learning model unit 320 may include various types of learning models for artificial intelligence, image analysis, and the like. For example, the learning model unit 320 may include a CNN or RNN model. The learning model unit 320 may change a preset (or previously stored) learning model.

The data processing optimization unit 330 may include and execute an optimization program or a tool for generating training data optimized for a specified learning model according to an embodiment of the present inventive concept. By applying a learning model to a small amount of medical data, a training result is received as a feedback and checked, and when there is an error, the error is corrected or amended and then the corrected data is reapplied to the learning model. When no error is found, the medical data is secured as training data.

The data processing optimization unit 330 according to an embodiment of the present inventive concept may include, for example, a data receiver and a manager (or a controller (e.g., a CPU and the like)), which are configured by a SW module, a HW module, or a combination thereof and receive a training result as a feedback.

FIGS. 4 and 5 respectively show a UI screen generated by the data processing optimization unit 330 according to an embodiment of the present inventive concept and displayed on the screen, and CT images on which marking is performed. In FIG. 4, a UI screen displayed on the screen may include some or all of a tab portion 400, an open image portion 410, a data adjustment portion 420, an addition and deletion portion 430, a marker or list display portion 440, and a viewer portion 450.

Here, the tab portion 400 may include different screens/functions depending on tabs. For example, different data annotation tasks may be possible, and may include an artificial intelligence/analysis model development function and a database function. The open image portion 410 may load 2D/3D image files in a folder. The data adjustment portion 420 may perform adjustments such as Slice (Data Sequences), Radius, Text Size, and the like. Slice is related to visualization of the previous/next image. Radius performs a movement and size adjustment of a marker (point) or a region of interest. Text Size may perform a font size adjustment. The addition and deletion portion 430 performs addition and deletion of a marker or a region of interest. The marker or (region of interest) list display portion 440 performs registration of a marker or a region of interest. The viewer portion 450 shows a 2D/3D image that is visualized.

FIG. 5 shows examples of annotation. A task may be performed by using various input devices (e.g., a keyboard, a mouse, a tablet, and the like that are usable) in a marker, region of interest annotation data generation, and a viewer, and an order may be assigned. In the viewer of FIG. 4, the region of interest annotation may be a task of assigning various positions, sizes, and shapes (e.g., a triangle, a rectangle, and the like).

The learning result output unit 340 may secure and store data optimized for a specific learning model through the learning model unit 320. For example, before the learning result output unit 340 secure training data, all training results by the learning model unit 320 are first provided to the data processing optimization unit 330 to correct errors, and then may be provided to the learning result output unit 340. However, training data including errors may be provided to the learning result output unit 340 through the learning model unit 320, and then the training data including errors may be erased upon the request of the learning model unit 320 and updated to the corrected training data.

As such, as the processing process of the training data output from the learning result output unit 340 may be performed in various ways, an embodiment of the disclosure may not be particularly limited to any one format. For example, in an embodiment of the disclosure, the training data processing process may include both of an erasure operation and a correction operation.

The data input unit 350 and the determination result output unit 360 mean that training is performed based on the training data generated and optimized according to the method according to an embodiment of the present inventive concept. The data output through the learning result output unit 340 may be provided to a device equipped with training data of a specific learning model, in detail, the same learning model engine as the learning model unit 320 of FIG. 3. Accordingly, it may be understood that the data is used to train new input data in the device and output a determination result.

FIG. 3 shows a data processing process according to an embodiment of the present inventive concept, and in an embodiment of the disclosure, the configuration of the training data generation device 120 of FIG. 1 is not particularly limited to the process as in FIG. 3.

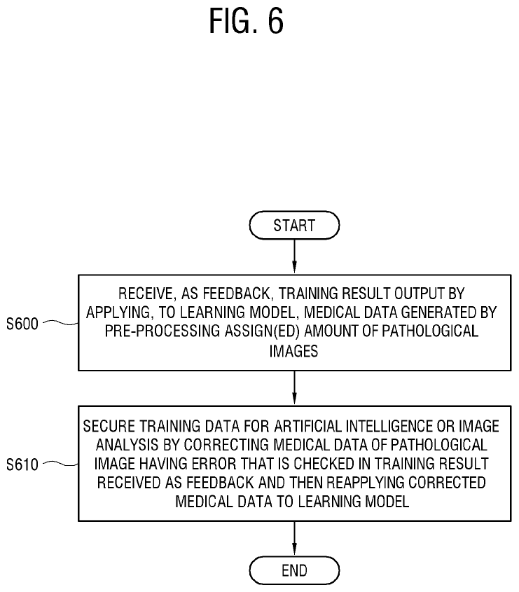

FIG. 6 is a flowchart of a driving process of the training data generation device of FIG. 1.

For convenience of explanation, referring to FIG. 6 with FIG. 1, the training data generation device 120 according to an embodiment of the present inventive concept receives, as a feedback, a training result output by applying, to a learning model, the medical data generated by pre-processing an assign(ed) amount of pathological images (S600).

Here, the pathological image may include a CT image and the like captured by various medical equipment. Furthermore, the pre-processing means that marking processing is performed on a portion such as a lesion, a tumor, or the like in the secured CT image. Information about a marking position may be referred to as a data set in the form of a coordinate value. For example, when marking is made with a total six points, the coordinate values of the points become a data set, and when an area is indicated by lines, the coordinate values of the lines or information about the characteristics of lines (e.g., color, thickness, and the like) become a data set.

Furthermore, the training data generation device 120 may secure (or generate) training data for artificial intelligence or image analysis by correcting the medical data of a pathological image having an error that is checked in the training result received as a feedback and then reapplying the corrected medical data to the learning model (S610).

For example, in an embodiment of the disclosure, assuming that training data for a total of ten thousand sheets of medical data is necessary, CT images are checked for a training result in units of 100 sheets, and when a result error is detected by checking, for example, an image processing value for a specific CT image, the error is corrected by correcting a data set related to the corresponding image. Accordingly, it may be seen that error-free training data is secured in the supervised learning method.

When the learning model is changed in the process, an operation of generating new training data optimized for the changed learning model by the method described above may be performed.

In addition to the above content, the training data generation device 120 of FIG. 6 may perform various operations, and as other detailed contents are sufficiently described above, descriptions thereof are replaced with the above descriptions.

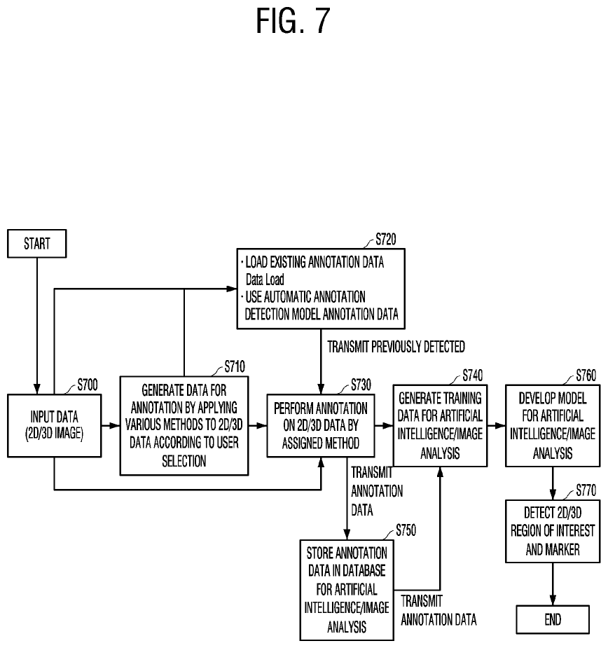

FIG. 7 is a flowchart of another driving process of the training data generation device of FIG. 1.

For convenience of explanation, referring to FIG. 7 with FIG. 1, the training data generation device 120 according to an embodiment of the present inventive concept generate data for annotation by collecting 2D and 3D data and applying various methods (e.g., marking and the like) to the collected data (S700 and S710).

Furthermore, the training data generation device 120 generates training data for artificial intelligence or image analysis by applying an optimization tool according to an embodiment of the present inventive concept (S720 to S750). In the process, when there is previously stored annotation detection data or model, after automatically applying the previously stored annotation detection data to new input data, and correcting the same, annotation data for the corresponding input data is generated and databased (S730 and S750).

By the above process, a model for artificial intelligence or image analysis may be developed (S760). For example, training data to be used therefor may be secured, and detection of a region of interest or a marker of the input data through the development model (S770). In other words, a determination result thereof may be output by performing training on the region of interest or a marker detection portion, based on the training data secured above in the S740.

In addition to the above content, the training data generation device 120 of FIG. 7 may perform various operations, and as other detailed contents are sufficiently described above, descriptions thereof are replaced with the above descriptions.

Although it has been described in the above that all the components of an embodiment of the disclosure are coupled as a single unit or coupled to be operated as a single unit, the disclosure is not necessarily limited to such an embodiment. Namely, within the purpose of the disclosure, one or more components among the components may be selectively coupled to be operated as one or more units. Also, although each of the components may be implemented as an independent hardware, some or all of the components may be selectively combined with each other, so that they may be implemented as a computer program having one or more program modules for performing some or all of the functions combined in one or more hardware units. Codes and code segments forming the computer program can be easily conceived by an ordinarily skilled person in the technical field of the disclosure. Such a computer program may implement the embodiments of the disclosure by being stored in a computer-readable medium, and being read and executed by the computer. Storage mediums for storing the computer program may include a magnetic recording medium, an optical recording medium, etc.

Here, a non-transitory readable recording medium means a medium capable of semi-permanently storing data and being readable by a device, not a medium for storing data for a short time, such as a register, cache, memory, and the like. In detail, the above-described programs may be stored and provided in a non-transitory readable recording medium such as CD, DVD, a hard disk, a Blu-ray disc, USB, a memory card, ROM, and the like.

As such, while the disclosure has been particularly shown and described with reference to preferred embodiments using specific terminologies, the embodiments and terminologies should be considered in descriptive sense only and not for purposes of limitation. Therefore, it will be understood by those of ordinary skill in the art that various changes in form and details may be made therein without departing from the spirit and scope of the disclosure as defined by the following claims.

### INDUSTRIAL APPLICABILITY

The present inventive concept is industrially applicable in the medical industry.

1. A training data generation device comprising:
a data receiver configured to receive, as a feedback, a training result output by applying, to a learning model, medical data generated by pre-processing an assigned amount of pathological images; and
a controller configured to secure training data for artificial intelligence (AI) or image analysis by correcting medical data of pathological images having errors checked based on the training result received as a feedback, and then reapplying the corrected medical data to the learning model.

2. The training data generation device of claim 1, wherein the controller is further configured to generate a data set for a position of a lesion marked on the pathological images for the pre-processing, and after correcting the generated data set, reapply the corrected data set to the learning model.

3. The training data generation device of claim 2, wherein the controller is further configured to perform automatic marking on the pathological images based on previously stored marking information, and correct a position by the automatic marking and apply the corrected position to the learning model.

4. The training data generation device of claim 2, wherein the controller is further configured to display, on a screen, a training result received as a feedback to make correction of the data set.

5. The training data generation device of claim 1, wherein the controller is further configured to secure new training data when the learning model is changed, by applying the secured training data to the changed learning model.

6. A method of driving a training data generation device, the method comprising:
receiving, by a data receiver, as a feedback, a training result output by applying, to a learning model, medical data generated by pre-processing an assigned amount of pathological images; and
securing, by a controller, training data for artificial intelligence (AI) or image analysis by correcting medical data of pathological images having errors checked based on the training result received as a feedback, and then reapplying the corrected medical data to the learning model.

7. The method of claim 6, wherein the securing of the training data comprises generating a data set for a position of a lesion marked on the pathological images for the pre-processing, and after correcting the generated data set, reapplying the corrected data set to the learning model.

8. The method of claim 7, further comprising performing, by the controller, automatic marking on the pathological images based on previously stored marking information, and correcting a position by the automatic marking and applying the corrected position to the learning model.

9. The method of claim 7, further comprising displaying, by the controller, on a screen, a training result received as a feedback to make correction of the data set.

10. The method of claim 6, further comprising securing, by the controller, new training data, when the learning model is changed, by applying the secured training data to the changed learning model.

11. A non-transitory computer-readable recording medium having recorded thereon a program for executing a method of driving a training data generation device,
the method of driving a training data generation device comprising:
receiving, as a feedback, a training result output by applying, to a learning model, medical data generated by pre-processing an assigned amount of pathological images; and
securing training data for artificial intelligence (AI) or image analysis by correcting medical data of pathological images having errors checked based on the training result received as a feedback, and then reapplying the corrected medical data to the learning model.
